(19) United States
(12) Patent Application Publication
(10) Pub. No.: US 2007/0275091 A1
(43) Pub. Date: Nov. 29, 2007

(54) Compositions And Methods For Improved Mucus Function

(76) Inventors: Malcolm King, Edmonton (CA); Gustavo J. Zayas, Edmonton (CA)

Correspondence Address: BERESKIN AND PARR, 40 KING STREET WEST, BOX 401, TORONTO, ON, M5H 3Y2, CA

(21) Appl. No.: 10/599,578

(22) PCT Filed: Mar. 30, 2005
(86) PCT No.: PCT/CA05/00463

§ 371 Date: Jun. 28, 2007

Related U.S. Application Data
(60) Provisional application No. 60/557,402, filed on Mar. 30, 2004.

(51) Int. Cl. A61K 31/715; A61K 33/14; A61K 33/22; A61K 33/24; A61K 38/00; A61P 43/00

(52) U.S. Cl. 424/617; 424/658; 424/663; 514/2; 514/59

(57) ABSTRACT

The invention relates to compositions and methods for enhancing mucus functions such as but not limited to improved respiratory tract mucus clearance. The compositions of the invention are mucothickening compositions. They can be used to improve mucus clearance in a subject in need thereof, such as in the treatment of conditions related to non-functioning cilia or to excessively thin mucus.

### FIELD OF THE INVENTION

This invention relates to compositions and methods for enhancing mucus functions and interactions, like improved respiratory tract mucus clearance, as well as mucus function in other systems of the body.

### BACKGROUND OF THE INVENTION

Infectious respiratory diseases are a prime cause of morbidity, mortality and health system utilization. Global health security has become broader and more complex and currently demands several measures to strengthen international control management capacity. Preparedness for the possible deliberate release of biological materials is currently among the highest concern worldwide. Questions have arisen regarding current strategies for protection of populations, and about the short and long-term capacity of public health infrastructures to respond to such events that pose catastrophic consequences.

Emerging epidemic-prone diseases, and the re-emergence of others is making this problem more complex. These pathogens can travel from country to country in different continents in a matter of hours, as exemplified by the recent Severe Acute Respiratory Syndrome coronavirus (SARS-CoV) outbreak. The SARS outbreak has validated these concerns in advance of the feared Influenza pandemic.

Currently several strategies and devices have been designed to protect many sectors of the society whether at peace time or during conflicts, to a range of infectious disease threats occurring naturally, or the possible deliberate use of biological agents. However, new, adequate and cost-effective control measures are definitively required.

The Severe Acute Respiratory Syndrome (SARS) caused by the SARS virus, is a coronavirus never before seen in humans. Since November 2002, when the first case of this new disease was detected, several thousand patients have been reported in more than 20 countries, with more than 438 cases and 44 deaths across Canada, as reported by the end of 2004. How SARS is transmitted remains to be clearly defined; however the group of researchers who identified the SARS virus considers that close person-to-person contact is the major way the disease spreads. They also acknowledge that diseases spread by droplets are the most difficult to control.

Since the majority of biological and chemical agents can enter the body through the respiratory system, it is a clear priority to protect the respiratory system during health and during disease. Since SARS is believed to be an airborne disease, it is critical to focus attention on the respiratory system. Two main complementary defense mechanisms protect the airways. Mucociliary clearance is the first line of defense and cough clearance is the backup or reinforcement if the first one is unable to carry out the task. Mucus plays a critical role in both clearance mechanisms (6).

Airborne diseases are transmitted when an infected person coughs, sneezes and perhaps even speaks.

When the airway mucus layer interacts with high-speed airflow as in coughing, there is formation of droplets of different sizes that are expelled to the surrounding environment as an aerosol. The concentration of droplets and their size distribution each plays an important role in transmission of airborne diseases or infections, such as SARS and other diseases such as influenza and tuberculosis.

Airway mucus derives from the goblet cells of the epithelial surface layer and the mucous cells of the submucosal glands. The mucous secretion is a non-homogeneous, viscoelastic fluid containing glycoproteins, proteins, and lipids in a watery matrix. The mucus along with serous fluid forms the airway surface fluid (ASF) that provides a protective milieu for the airways. The composition and physical characteristics of ASF allow for normal ciliary activity and airway protection (6). When disruption of normal secretory or mucociliary clearance processes occurs, respiratory secretions can accumulate and impair pulmonary function, reduce lung defenses and increase the risk for infection and possibly neoplasia (14-16).

Mucomodulator therapy—changing the physical properties of ASF—is designed to enhance the clearance of mucus from the respiratory tract as well as to optimize aspects of lung defense that depend on the mucus layer. Mucomodulators include mucolytics, designed to disrupt the structural macromolecules that give respiratory tract mucus its physical characteristics, and other agents designed to increase mucus flow by stimulating ciliary activity or improving periciliary fluid hydration. Mucomodulator therapy combating mucus retention is a major consideration in the treatment of cystic fibrosis and other chronic lung diseases in which mucus hypersecretion and impaired airway clearance produce symptoms (17,18).

Mucus factors affecting mucus clearance rate (MCR) are the mucus depth and mucus viscoelastic properties (6,12). When the mucus layer is too thick, and clearance by the cilia is hindered, clearance by coughing takes over. Mucus needs to be both viscous and elastic. The elasticity of mucus is important for clearance by cilia because it efficiently transmits the momentum of the cilia during their forward stroke without energy loss. The viscosity of mucus results in energy loss, but it is necessary so that mucus can be displaced and either expectorated or swallowed. A balance between these factors must be maintained for optimal MCR.

Elasticity in respiratory tract mucus also retards wave formation and two-phase flow during coughing. Thus, mucus that is elastic may be efficient in mucociliary clearance, but inefficient in cough clearance (13), and thus a dynamic balance between mucus viscosity and elasticity may be determined by nature. The effects of mucolytic treatments on both forms of clearance should be considered in evaluating their efficacy.

It should be noted that the viscoelasticity relevant to cough clearance is not necessarily the same as the viscoelasticity relevant to mucociliary clearance. Mucociliary clearance is defined by low frequency, low amplitude conditions, while cough clearance is governed by high frequency, high amplitude conditions (6,13). While there is generally an association between the low and high frequency/amplitude forms of viscoelasticity, it is possible to dissociate these parameters to some degree. Thus it is conceivable to design or modify mucus to show elastic behavior at low frequency/amplitude (predicting good mucociliary clearability) and less elastic behavior at high frequency/amplitude (predicting also good cough clearability).

There is a need for pharmacological interventions aimed at modulating the physical and biochemical characteristics of mucus secretions, for example to minimize aerosolization of expectorated material carrying an infection in respiratory secretions. There is also a need to minimize adverse effects of modulating the physical and biochemical characteristics of mucus secretions on mucociliary clearance. There is also a need to improve mucus clearance, such as mucociliary clearance and cough clearance (expectoration).

### SUMMARY OF THE INVENTION

The transmission of an airborne disease requires a) the transmissor or source (an individual with the disease), b) the surrounding environment and c) the recipient (a non-infected individual). Knowledge of the dynamic that takes place among the transmissor (e.g. a SARS patient), the recipient (e.g a healthy individual) and the surrounding microenvironment between them, as well as the aerosolization that is required for transmission of airborne diseases, will provide information about the mode of transmission. Mucomodulation will reduce or prevent the spread of airborne diseases from the transmissor, as well as enhance the protective function of the mucus barrier in the recipient, when administered to these respective individuals.

As such, in one aspect, the present invention includes the administration of a mucomodulator to a non-infected subject to enhance the functions of the layer of mucus anywhere in the body, for example, but not limited to, the protective function of the respiratory mucus layer. Another aspect of the invention is the administration of a mucus thickening agent to a subject to inhibit, prevent and/or treat a medical condition. In another aspect, the present invention relates to the inhibition of transmission of a medical condition.

Accordingly, the present invention includes a method of enhancing mucus function comprising administering an effective amount of a mucothickening agent to a subject in need thereof. The invention also includes a use of a mucothickening agent to enhance mucus function as well as a use of a mucothickening agent to prepare a medicament to enhance mucus function. In an embodiment of the invention, the mucus function is selected from respiratory track mucus clearance, epithelial protection, ion exchange and nutrient intake. In an embodiment of the invention, the respiratory track mucus clearance is mucociliary clearance and/or cough clearance.

For example the methods and compositions of the present invention can be used to improve physical and/or biochemical properties of the layer of mucus lining the respiratory system, digestive system, urinary tract and/or the reproductive system. For example, they can be used to enhance the protective function of the layer of mucus in the above mentioned systems. They can also be used to improve mucus properties of the digestive system that participate in, for example, but not limited to, nutrient intake processes, epithelium protection and drug absorption. They can also be used to improve mucus properties of the reproductive system to enhance conception and/or reduce, suitably inhibit, more suitably prevent the transmission of sexually transmitted diseases (STD), such as, but not limited to viral, fungal and/or bacterial agents.

Also included within the scope of the present invention is a method of inhibiting aerosolization and/or transmission of an airborne disease comprising administering an effective amount of a mucothickening agent to a subject in need thereof. The invention also includes a use of a mucothickening agent to inhibit aerosolization and/or transmission of an airborne disease and a use of a mucothickening agent to prepare a medicament to inhibit aerosoiization and/or transmission of an airborne disease. In an embodiment of the invention, the airborne disease is selected from, but not limited to, SARS, tuberculosis and influenza. In a further embodiment, the method of inhibiting aerosolization and/or transmission of an airborne disease occurs by decreasing aerosolizable respiratory secretions in a way to minimize any adverse effect on mucus clearance, optionally in combination with enhancement of mucus clearance. Mucus clearance can be mucociliary clearance and/or cough clearance (expectoration).

The present invention also includes a method of treating a condition related to thin mucus or cilia malfunction or non-function, comprising administering an effective amount of a mucothickening agent to a subject in need thereof. The invention also relates to a use of a mucothickening agent to treat a condition related to thin mucus or cilia malfunction or non-function and a use of a mucothickening agent to prepare a medicament to treat a condition related to thin mucus or cilia malfunction or non-function. Conditions related to thin mucus or cilia malfunction or non-function include any condition of impaired airway secretion management, including, but not limited to, cystic fibrosis, asthma, chronic obstructive pulmonary disease (COPD), spinal chord injury, neuromuscular disease and those conditions or diseases requiring assisted mechanical ventilation like patients in an intensive care unit (ICU).

The methods and compositions of the invention can be used to improve physical and/or biochemical properties of the layer of mucus lining the respiratory system, digestive system, urinary tract or the reproductive system. For example, they can be used to enhance the protective function of the layer of mucus in the above mentioned systems. They can also be used to improve mucus properties of the digestive system that participate in, for example, but not limited to, nutrient intake processes, epithelium protection and drug absorption. They can also be used to improve mucus properties of the reproductive system to enhance conception and/or reduce, suitably inhibit, more suitably prevent the transmission of sexually transmitted diseases (STD), such as, but not limited to viral, fungal and/or bacterial agents.

The invention further includes a method of diagnosing a subject with a medical condition comprising administering an effective amount of mucothickening agent to said patient and screening said mucus for said medical condition. The invention also includes a use of a mucothickening agent to diagnose a subject with a medical condition as well as a use of a mucothickening agent to prepare a medicament to diagnose a subject with a medical condition.

The mucothickening agent is any agent that promotes the formation of one or more of the following in mucus: covalent bonds, ionic bonds, hydrogen bonds, van der Waals' forces, intermingling or extracellular DNA & F-actin network. In an embodiment of the invention, the mucothickening agent is selected from: high molecular weight polysaccharides, such as dextran, tetrafunctional anions, such as sodium tetraborate, salts of divalent cations, such as calcium or magnesium chloride, and polycationic agents, such as polylysine, polyarginine or polymyxin B, and pharmaceutically acceptable salts thereof (where applicable). In another embodiment, the mucothickening agent is one or more of the said agents.

The present invention further includes pharmaceutical compositions comprising a mucothickening agent suitable for use in the methods of the present invention, and pharmaceutically acceptable carriers or diluents.

The administration of a mucothickener to a subject using the methods of the present invention optimizes the defensive properties of the layer of mucus in a non-infected subject which leads to inhibition, reduction or minimization of transmission of medical conditions caused by injury agents or airborne infections when exposed to the subject. Further, the administration of a mucomodulator to a subject enhances cohesiveness, thickness and/or immunologic defense properties of the mucus layer in a non-infected subject so that these layers may trap and then expel injury agents and, thereby, inhibit the penetration of such agents into the body. Still further, the administration of a mucomodulator to a subject enhances mucus clearance, which is a major consideration in conditions of impaired airway secretion management.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 is a diagram of the various types of bonds occurring in respiratory tract mucus.

FIG. 2 shows bar graphs illustrating the effect of dextran molecular weight on the viscoelasticity (A) and spinnability (B) of respiratory tract mucus of cystic fibrosis patients.

FIG. 3 shows scanning electron micrographs illustrating the effect of CaCl₂ on the mucus from the frog palate. FIG. 3A is the frog mucus treated with Frog Ringers alone and FIG. 3B is with CaCl₂. In FIG. 3B the ciliated surface of the palate can be seen below the mucus and does not appear different to control views.

FIG. 4A is a bar graph illustrating the effect of the mucomodulator CaCl₂ (mucomodulator C) on mucociliary clearance in the frog palate model. Treatment with CaCl₂ did not alter the mucus transit time.

FIG. 4B is a graph illustrating the effects of five different mucomodulators (mucomodulators A, B, C, D and PB) over the concentration range 10⁻⁸ (XL-8) to 10⁻¹ M (XL-1) on mucociliary clearance velocity in the frog palate model. The mean mucociliary clearance velocity for Frog Ringers is also shown.

FIG. 5 shows bar graphs illustrating the effect of increasing amounts of mucomodulator B (sodium tetraborate) on (5A) mucociliary clearance in the frog palate model and (5B) cough clearance (expectoration) in a simulated cough machine.

FIG. 6A is a bar graph illustrating the effect of increasing amounts of added sodium tetraborate (mucomodulator B) on mucus simulant spinnability.

FIG. 6B depicts target bullseye results showing the effect of sodium tetraborate (mucomodulator B) on the aerosolization and dispersion of synthetic mucus in a cough simulator.

FIG. 7 is a graph illustrating the effect of saline, sodium tetraborate (mucomodulator B) and high molecular weight dextran (mucomodulator D) on tracheal mucociliary clearance in anesthetized dogs as a percent of control.

### DETAILED DESCRIPTION OF THE INVENTION

Definitions

The expression “enhancing mucus function” as used herein means increase, improve, and/or strengthen the natural capacity of mucus, anywhere in the body, to provide protection to the epithelium, hence to the body, as compared to a control or as compared to the degree of protection in a subject that was not administered an effective amount of mucomodulator, such as the mucothickening agent, in accordance with the method of the invention.

“Inhibiting aerosolization” as used herein means reducing the amount of aerosolization (size and number of droplets in the aerosol) as compared to a control or as compared to the degree of aerosolization in a subject that was not administered an effective amount of mucomodulator, such as a mucothickening agent, in accordance with the method of the invention.

“Minimizing the adverse effect on mucus clearance” as used herein means reducing the degree of difference in mucus clearance in a subject receiving the treatment of the present invention (administration of a mucomodulator, such as a mucothickening agent) versus a control or one that does not receive such treatment or some other selected baseline.

“Enhancing” or “improving” mucus clearance, as used herein means increasing mucus clearance as compared to a control or a subject that does not receive the treatment of the present invention or some other determined baseline.

“Mucomodulation” as used herein means modifying the physical properties of mucus (viscoelasticity, cohesivity, surface tension) by altering the viscosity and/or elasticity.

“Mucomodulator” as used herein means an agent designed or intended to produce mucomodulation of mucus, for example, but not limited, to enhance the clearance of mucus from the respiratory system and/or optimize aspects of lung defense that depend on the mucus layer.

“Mucothickening agent” as used herein means a mucomodulator designed or intended to produce a thicker mucus

“Thick” as used herein means viscous and/or elastic and/or rigid, in reference to the physical properties of mucus. In more scientific terms, “thick” means of high modulus of viscoelasticity (the capacity to deform and flow under applied pressure). “Thin” will have a converse meaning.

Thick mucus, such as seen in adult patients with cystic fibrosis, would have an elastic modulus in the range of 195-1780 dyn/cm² and a viscosity in the range of 7.4-70 Poise, when measured at a frequency of 10 radians per second in a magnetic rheometer (4). Normal mucus, as sampled from the trachea of healthy, non-infected volunteers by bronchoscopy, exhibits an elastic modulus in the range 107-490 dyn/cm² and a viscosity in the range 3.8-17.4 Poise (2). Thin mucus has lower elastic modulus and viscosity than normal. See appended sputum reference data in Table 1.

“Respiratory tract mucus” as used herein means mucus lining the respiratory epithelium, including the nasal passages and the tracheobronchial airways.

“High molecular weight dextran” as used herein means mean molecular weight ca. 70,000 Daltons or greater, more suitably in the range of 100,000-1,000,000 Daltons, as assayed by conventional viscometric techniques.

The term “pharmaceutically acceptable” as used herein means compatible with the treatment of human or veterinary subjects.

The term “pharmaceutically acceptable salt” means an acid or basic addition salt (wherein appropriate) which is suitable for or compatible with the treatment of human or veterinary subjects.

The term “pharmaceutically acceptable acid addition salt” as used herein means any non-toxic organic or inorganic salt of any base mucothickening compounds of the invention. Basic compounds of the invention that may form an acid addition salt include those having a basic nitrogen, for example NH₂ and NHC₁₋₄alkyl. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of the compounds of the invention are more soluble in water and various hydrophilic organic solvents, and generally demonstrate higher melting points in comparison to their free base forms. The selection of the appropriate salt will be known to one skilled in the art. Other non-pharmaceutically acceptable salts, e.g. oxalates, may be used, for example, in the isolation of the compounds of the invention, for laboratory use, or for subsequent conversion to a pharmaceutically acceptable acid addition salt.

The term “pharmaceutically acceptable basic addition salt” as used herein means any non-toxic organic or inorganic base addition salt of any acid mucothickening compound of the invention. Acidic compounds of the invention that may form a basic addition salt include, for example, those substituted with a group having acidic hydrogen, for example C(O)OH. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline or ammonia. The selection of the appropriate salt will be known to a person skilled in the art. Other non-pharmaceutically acceptable basic addition salts, may be used, for example, in the isolation of the compounds of the invention, for laboratory use, or for subsequent conversion to a pharmaceutically acceptable acid addition salt.

The formation of a desired compound salt is achieved using standard techniques. For example, the neutral compound is treated with an acid or base in a suitable solvent and the formed salt is isolated by filtration, extraction or any other suitable method.

The term an “effective amount” or a “sufficient amount” of an agent as used herein is that amount sufficient to effect beneficial or desired results, including clinical results, and, as such, an “effective amount” depends upon the context in which it is being applied. For example, in the context of administering an agent that enhances mucus function, an effective amount of an agent is, for example, an amount sufficient to achieve such a enhancement in mucus function as compared to the response obtained without administration of the agent.

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

“Palliating” a disease or disorder means that the extent and/or undesirable clinical manifestations of a disorder or a disease state are lessened and/or time course of the progression is slowed or lengthened, as compared to not treating the disorder.

The term “subject” as used herein includes all members of the animal kingdom. In an embodiment of the invention, the subject is a human.

### METHODS OF THE INVENTION

The present invention relates to a novel mucotherapy intended to modulate the physical characteristics of viscoelasticity, cohesivity and surface tension of the respiratory secretions to enhance the natural normal protective capability of the layer of mucus in health and in disease, and to minimize the aerosolization that is required for transmission of airborne diseases. The invention does the opposite of what mucolytic agents traditionally do. In this aspect, the invention includes methods and compositions to reduce the aerosolizability of respiratory secretions while maintaining mucociliary clearability, and thus normal airway clearance function. This requires more subtle manipulation of mucus viscosity and elastic properties than conventional mucolytic therapies offer (11). Ninety-five percent of mucus is water and the remaining 5% is made up of proteins, carbohydrates, salts, etc. Therefore, when the airway mucus layer interacts with high-speed airflow during coughing, there is formation of droplets of different sizes that are expelled to the surrounding environment as aerosol. The concentration of droplets and their size distribution may each play an important role in transmission of epidemiologically and clinically important airborne diseases like SARS or the flu when an infected person coughs or sneezes, and even possibly when speaking.

The number of infective agents (species) released to the environment are related to the amount expectorated, and the droplet size will determine their fate. Micron-sized droplets dry quickly and can remain airborne for long periods and possibly reach many persons through the environment via a common system: ventilation, water, heating. In contrast, larger droplets are propelled onto the nearest surface: respiratory, orogastrointestinal or ocular mucosas of a nearby person, or settle readily to the floor. Regarding transmission, larger particles may have an immediate effect—direct but limited action—while micron-size particles may have more indirect effects with longer-lasting and widespread consequences. The present invention also addresses the question of which is the proper balance in modulating the size of the droplets.

The methods of the invention involve an increase in the crosslinking binding sites in the mucin glycoprotein gel network, thereby raising mucin gel viscoelasticity and/or forming poorly soluble mucin complexes. The result is a less aerosolizable respiratory secretion, which decreases the degree of contiguousness.

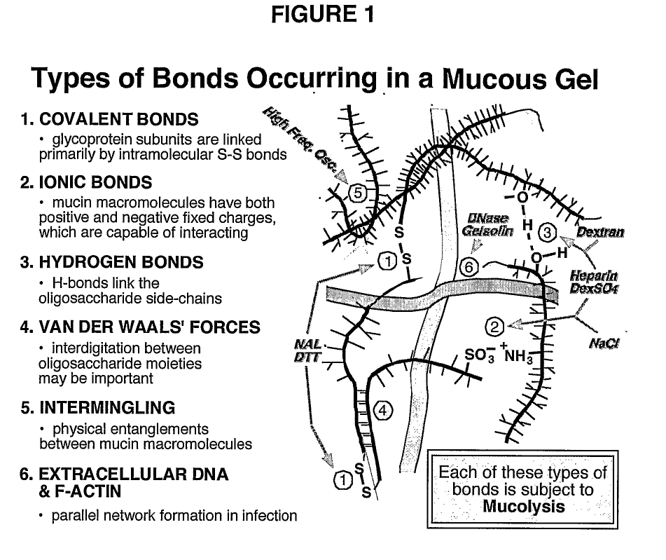

The mucus macromolecule consists of a protein core surrounded by short oligosaccharide side-chains, held together by different links: O-glycosidic bonds, disulphide bridges, hydrogen bonds and ionic bonds. These links are the targets of the existing mucolytic agents (11). The bonds that keep mucus together and effect viscoelasticity are depicted in FIG. 1 and include: covalent bonds, ionic bonds, van der Waals' forces, intermingling, and extracellular DNA & F-Actin

Breaking covalent bonds reduces mucin molecular weight and results in changes to both mucociliary and cough clearability, as predicted by viscoelasticity measurements. Further, the reverse mucolyis of the present study involves the use of nontoxic treatments. For example, glutaraldehyde will increase mucus crosslink density, but this would be toxic. Disrupting ionic and/or hydrogen bonds produce more subtle effects on viscoelasticity, since only side-chain interactions are affected. The approaches include increasing ionic interactions by adding divalent cations, increasing H-bond crosslinking with agents like high molecular weight dextran or other high MW polysaccharides, and increasing specific interactions between side chain sugars, as with sodium tetraborate or other tetrafunctional anions which will selectively crosslink galactose units.

As such, a number of different mucothickening agents can be used in the invention that target the same or different bonds, noted above. These could be applied alone or in combination, in order to enhance mucociliary clearability, while reducing aerosolizability of the secretions.

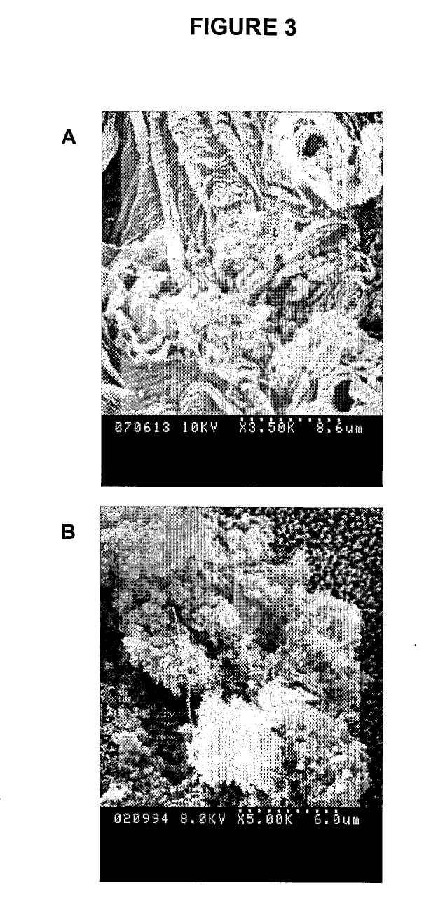

In an embodiment of the invention, the mucothickening agent is one that increases the concentration of divalent cations in the mucus through the administration of, for example, divalent cation salt solutions, such as calcium and/or magnesium salt solutions. Conceptually, this is the opposite of what occurs in nature during mucin exocytosis, where intracellular mucin granules which were held tightly through calcium ion crosslinks give way to much looser interactions as sodium ions exchange for calcium during fusion with the apical membrane. An example of the use of this method is shown in the experiments illustrated in FIG. 3. In this example, using the ex-vivo frog palate model, an increase in mucus clumping with calcium ion administration while maintaining mucociliary clearance was observed.

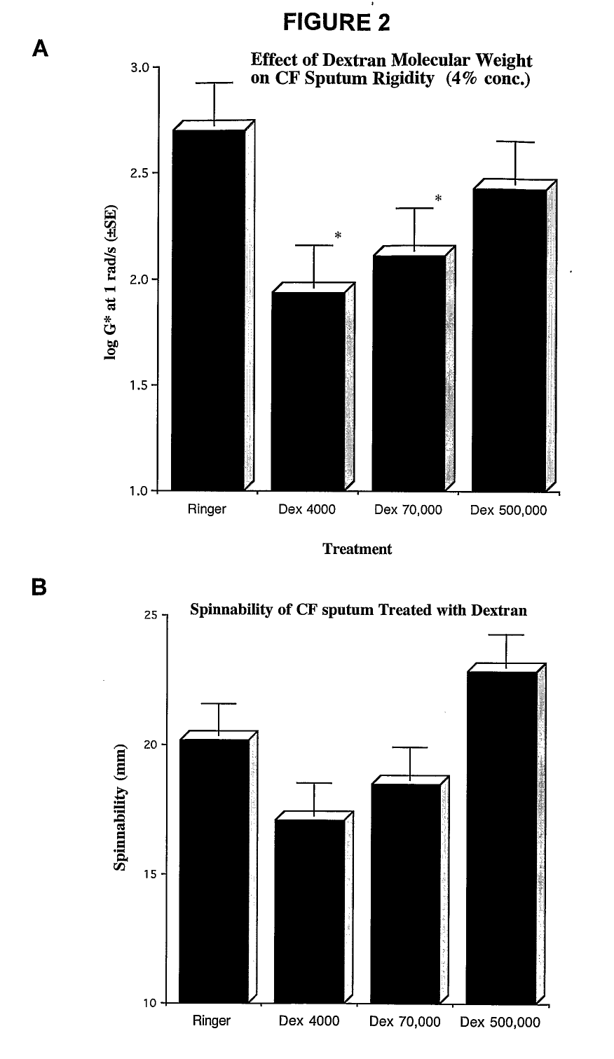

In a second embodiment of the invention, the mucothickening agent is to a high molecular weight polysaccharide, such as high molecular weight dextran (ca. 500,000 MW) or a pharmaceutically acceptable salt thereof. The use of low molecular weight dextran as a mucolytic agent has been developed by King and collaborators (7). This therapy has passed a phase I trial, and is currently in a phase 2 clinical trial in patients with cystic fibrosis. Low molecular weight dextran serves to reduce mucin gel crosslinking by disrupting intermolecular mucin-mucin H-bond crosslinks, substituting instead mucin-dextran crosslinks which are dysfunctional for network formation. On the other hand, high molecular weight (HMW) dextran has approximately the same molecular weight as the subunits of mucin macromolecules; in this case mucin-dextran crosslinks are approximately as effective as the original mucin-mucin crosslinks (FIG. 2). Interestingly, HMW dextran tends to raise elasticity relative to viscosity (as indicated by the increase in spinnability relative to log G*), thus its use would tend to inhibit aerosolizability, which will depend on spinnability, while maintaining mucociliary clearability. Surprisingly, it has been found to also improve or increase respiratory tract mucus cough clearability.

In a third embodiment of the present invention the mucothickening agent is a pharmaceutically acceptable salt of a tetrafunctional anion such as, but not limited to, sodium tetraborate, which causes reversible crosslink formation between galactose units, the major neutral sugar component of mucins. In model studies using vegetable polysaccharides, sodium tetraborate preferentially raises elasticity relative to viscosity, and would favour mucociliary clearability at the expense of cough clearability and aerosolizability.

In a fourth embodiment of the invention, the mucothickening agent is a pharmaceutically acceptable salt of a polycationic agent, such as, but not limited to polylysine, polyarginine or polymyxin B.

The methods of the present invention provide several benefits for the patients, for the caregiver and also for the general population. The mucothickeners of the present invention would enhance containment in an outbreak and would allow more time to make studies and adjustments in the strategies designed to reduce casualties. The route of administration of the mucothickeners of the present invention is suitably by, but not limited to, inhaled aerosols dispensed by affordable mask inhalers or other types of aerosol administration devices known in the art.

The transmission of SARS and similarly transmitted diseases can be controlled by implementing a pharmacological intervention aimed at modulating the physical and biochemical characteristics of the respiratory secretions in order to minimize expectorated material that carries the infection.

Applications

The methods and compositions of the invention can be used to improve the normal and natural functions (for example, but not limited to, protective, immunological defenses and/or ion exchange) of the layer of mucus anywhere in the body's system, including the respiratory, digestive, reproductive and/or urinary tract systems. The methods and compositions of the present invention can also be used to modulate the physical properties of mucus to enhance airway mucus by adding crosslinks in a selective and controlled manner. They can also be used to reduce mortality and improve quality of life in individuals who have difficulties with airway secretions management.

The methods and compositions of the present invention can also be used to increase cohesive interactions between mucin macromolecules to facilitate mucus clearance, most likely by airflow dependent mechanisms. It can also be used to benefit many, if not most patients with chronic airway diseases who depend in whole or in part of airflow clearance mechanisms to maintain airway hygiene. The methods and composition of the invention might be of particular importance for those with conditions related to thin mucus or cilia malfunction or non-function, including but not limited to, any condition of impaired airway secretion management, including, but not limited to Chronic Obstructive Pulmonary Disease (COPD), spinal chord injury, neuromuscular disease and those conditions or diseases requiring assisted mechanical ventilation like patients in an Intensive Care Unit (ICU). It can also be used in mucus plugging conditions like, but not limited to, asthma and/or cystic fibrosis.

The methods and compositions of the present invention can be used in a range of infectious disease threats occurring naturally like the feared influenza pandemic or the possible deliberate use of biological agents. It can also be used in veterinarian conditions in mammalian and/or non-mammalian animals.

As stated above, the methods and compositions of the invention can be used to improve physical and/or biochemical properties of the layer of mucus lining the respiratory system, digestive system, urinary tract or the reproductive system. For example, they can be used to enhance the protective function of the layer of mucus in the above mentioned systems. They can also be used to improve mucus properties of the digestive system that participate in, for example, but not limited to, nutrient intake processes, epithelium protection and drug absorption. They can also be used to improve mucus properties of the reproductive system to enhance conception and/or reduce, suitably inhibit, more suitably prevent the transmission of sexually transmitted diseases (STD), such as, but not limited to viral, fungal and/or bacterial agents.

The method and compositions of the invention can be used to modulate the viscoelasticity of respiratory tract mucus. They can be used to thicken said respiratory secretions to minimize the aerosolization of virus or bacteria-containing secretions during cough or sneezing. They can also be used to minimize any adverse effect on mucociliary clearance or even better to improve mucus clearance, such as mucociliary clearance and/or cough clearance. They can also be used to thicken said respiratory secretions to enhance the protective effect of the respiratory mucus layer against uptake of airborne pathogens in the recipient. As such the methods and compositions of the invention can be used to treat conditions where the mucus is too thin or where cilia is non-functioning. It can also be applied to thicken the mucus layer in other parts of the body such as the digestive and/or the genito-urinary systems where additional protection against pathogens would reduce the risk of infection. (e.g. respiratory or non-respiratory tract mucus). Such diseases include but are not limited to bronchiectasis and some types of chronic bronchitis, STD, diarrhea, by administering a therapeutically effective amount of said mucothickening agent to a patient in need thereof.

In another embodiment, the methods and compositions of the invention can be used to reduce, suitably to inhibit, more suitably to prevent, the transmission of clinically and epidemiologically important pathogens in airborne transmissible conditions, such as, but not limited to SARS, influenza, and tuberculosis. The invention works by entrapping said viral or bacterial particles in a mucus that permits the mucus to be expectorated from the lungs (e.g. 10 microns or greater, above the normal respirable range), but minimizes formation of small viral or bacterial containing particles (e.g. 2 microns or less), that can aerosolize upon coughing and remain suspended for a period of time, and thus be spread more widely.

The methods and compositions of the invention can also be used to obtain a mucus sample and analyze its contents for diagnosis of medical conditions e.g. by screening of viral and bacterial particles or cells in said mucus using screening methods known in the art, to determine whether the patient has a mucoviscoelastic-related condition (too thick or too thin), to determine appropriate dosage range of the mucomodulator, suitably mucothickening agent. Said dosage range can be determined by taking a mucus sample, subjecting it to various dosages of mucomodulator, and then, by assessing the effect of said mucomodulator on the viscosity of said mucus, determining the suitable dosage of said mucomodulator to get the desired mucomodulatory effect. Suitably said mucomodulator is a mucothickening agent and the effect is to thicken the mucus.

Pharmaceutical Compositions

The above-described mucomodulators may be formulated into pharmaceutical compositions for administration to subjects in a biologically compatible form suitable for administration in vivo. By “biologically compatible form suitable for administration in vivo” is meant a form of the substance to be administered in which any toxic effects are outweighed by the therapeutic effects. The substances may be administered to living organisms including humans, and animals.

Administration of a therapeutically effective amount of pharmaceutical compositions of the present invention is defined as an amount effective, at dosages and for periods of time necessary to achieve the desired result. For example, a therapeutically active amount of a substance may vary according to factors such as the disease state, age, sex, and weight of the individual, and the ability of the substance to elicit a desired response in the individual. Dosage regimes may be adjusted to provide the optimum therapeutic response. For example, several divided doses may be administered daily or the dose may be proportionally reduced as indicated by the exigencies of the therapeutic situation.

An active substance may be administered in a convenient manner such as by direct application to, but not limited to, the respiratory tract mucus, e.g. by instillation, by aerosol spray, and/or by other means such as injection (subcutaneous, intravenous, etc.), but most suitably by inhalation, such as through an inhaler or respiratory mask. Depending on the route of administration, the active substance may be coated in a material to protect the compound from the action of enzymes, acids and other natural conditions that may inactivate the compound. It may be delivered as a nebulized solution or suspension in an appropriate vehicle or as a dry powder formulation, using an appropriate breath-actuated device.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (2003-20th edition) and in The United States Pharmacopeia: The National Formulary (USP 24 NF19) published in 1999. On this basis, the compositions include, albeit not exclusively, solutions of the active substances in association with one or more pharmaceutically acceptable carriers or diluents, and may be contained in buffered solutions with a suitable pH and/or be iso-osmotic with physiological fluids. In this regard, reference can be made to U.S. Pat. No. 5,843,456. As will also be appreciated by those skilled, administration of substances described herein may be by an inactive viral carrier.

With regard to administration by inhalation, suitable preparations can be prepared in accordance with methods known in the art. (See for example references 5, 9 and 10).

In one embodiment of the invention, a 10-100 mmol solution of CaCl₂ is administered to effect “clumping” while still maintaining mucociliary clearance. In another embodiment, sodium tetraborate is administered at a 1-10 mmol concentration to an effect the addition of crosslinks to galactose-containing macromolecules. In yet another embodiment, high molecular weight dextran is administered at about the 4% concentration range, while poly-L-arginine can increase mucus crosslinking at a concentration of less than 1%. In one suitable embodiment, the neutral polysaccharides and cationic polymers are administered by means of dry powder inhalers, and thus the target concentration will be the pure material, i.e. 100%. In another embodiment, calcium and borate solutions are delivered by conventional wet nebulizers.

Kits

The reagents suitable for carrying out the methods of the invention may be packaged into convenient kits providing the necessary materials, packaged into suitable containers. Such kits may include all the reagents required to perform the method of the invention, e.g., the mucomodulator, and optionally the inhaler or other physical device used for administration of the mucomodulator, and optionally a set of instructions for using the kit and components thereof in the methods of the invention. The kit may also include muco-collective devices to collect mucus samples for carrying out diagnostic test and assessments of the invention.

The following non-limiting examples are illustrative of the present invention.

### EXAMPLES

### Methods

Mucus Viscoelasticity:

Mucus can be considered a viscoelastic fluid, since it exhibits both liquid-like (viscous) and solid-like (elastic) properties (1). Viscosity is the resistance to flow and represents the capacity of a material to absorb energy as it moves. Elasticity is the capacity of a material to store the energy used to move or deform it. The measures of viscosity and elasticity are dependent on both the frequency and amplitude of measurement, as well as the type of measurement device (6). The relative proportions of elasticity and viscosity are as important in describing how a material such as mucus behaves when it is subjected to external forces, as are the absolute values of either parameter by itself.

The magnetic microrheometer technique is used to measure the viscosity and elasticity of mucus (19). A 100 μm steel ball is positioned in a 5-10 mg sample of mucus, and the motion of this sphere under the influence of an electromagnet is used to determine the rheological properties of the mucus. The image of the steel ball is projected via a microscope onto a pair of photocells, whose output is amplified and transmitted to a digital storage oscilloscope. By plotting the displacement of the ball against the magnetic driving force, the viscoelastic properties of the mucus can be ascertained. Two derivative parameters—mucociliary clearability index (MCI) and cough clearability index (CCl)—are computed from in vitro relationships (King M. Role of mucus viscoelasticity in cough clearance. Biorheology 1987; 24: 589-597).

Cough Clearability Assay:

The simulated cough machine system comprises the following elements: A 10-L tank with compressed air, which serves as a pressure reservoir that generates airflow, simulating the lungs during a cough maneuver. The pressure generated prior to a normal adult cough is approximately 8 psi pressure. This pressure value was used to simulate the airflow pattern of a human cough for each trial.

The model “trachea” is a 28 cm long rigid acrylic tube with a circular 2.5 cm cross-section. It has an 8 cm removable portion to allow us to measure the weight of the sample of mucus simulant loaded before and immediately after each “cough” maneuver. A solenoid valve located between the pressure reservoir and the model trachea control the gas released from the tank.

An aliquot of sputum or mucus simulant is layered on the bottom of the model trachea and driven forward by the pressurized gas. The distance traveled by sputum samples under standardized cough-simulating airflow is used as a measure of cough clearability. The simulated cough maneuver is repeated four times with each sputum sample. Displacement of the MS sample loaded, expressed in millimeters, is measured from the initial placement to the farthest point with a visible bulk concentration of mucus.

Airway clearance (“expectoration” of mucus simulant from the “airways”): Measurements of the weight of the MS sample loaded before and after each cough maneuver were carried out in a Mettler AE 163 microbalance.

The depth of mucus and the airflow linear velocity are critical determinants of cough clearance. Mucus physical properties that are important to cough clearance are the viscosity of the mucus, and the elastic component. This latter component impedes forward motion and results in recoil after the cough event. Cohesivity allows mucus to hold together, and contributes to the surface properties, both on the air-mucus interface, as well as at the interface with the periciliary layer. Mucus that is elastic may be efficient in mucociliary clearance, but inefficient in cough clearance. The dynamic balance between mucus viscosity and elasticity may be altered in airway pathologies associated with mucus.

Frog Palate Preparation:

From a bullfrog, Rana catesbiana, the upper portion of the head is removed modifying the procedures described in previous works. This is done by pithing the frog after lowering the body temperature to abolish pain sensation, and then cutting with scissors through from the junction of the posterior pharynx and esophagus out to the skin of the back. The palate is checked for macroscopic lesions, such as ulcers, or evidence of inflammation. The palate is then placed inside the frog chamber, a wooden box with a glass top and fitted glass front and manipulated through glove openings, and viewed under a dissecting stereomicroscope provided with a reticulated eyepiece. Humidity inside the box is maintained at 100% using a Pari nebulizer; the box is maintained at room temperature (22° to 24° C.). Before carrying out any measurement, the palate is allowed to stabilize inside the box for 15 minutes.

Mucus Transport Velocity (MTV):

Mucociliary clearance is determined by observing the movement of particles of charcoal powder gently deposited on a sample of mucus on the palate surface; its clearance is visually monitored and hence MTV determined. The displacement of a 1-5 μL of endogenous frog mucus sample is timed as the trailing edge moves across a predetermined segment. MTV is calculated by dividing the distance traveled by the time elapsed, based on at least five measurements of the time required for the mucus sample to travel the defined distance (3).

Cilia Beat Frequency:

Studies on cilia beat frequency in situ on the excised fresh frog palate are carried out to assess the earliest effects of the agent being tested (8). These studies involve the use of a microscope acquisition system capable of acquiring digital video images of beating cilia on the surface of the excised frog palate. The action of the cilia can be correlated with other parameters of mucus transport for an enhanced understanding of the role of the cilia in mucociliary transport in health and disease.

Scanning Electron Microscopy:

Samples of mucus and tissue are placed in 2.5% glutaraldehyde solution immediately after collection and stored at 4° C. until processing. Briefly, the samples are post-fixed in 1% osmium tetroxide in Millonig's buffer at room temperature for one hour. They are then washed briefly in a series of ethanol (50-100%), ten minutes at each step, followed by two additional periods of absolute ethanol (10 minutes each). The samples are further dehydrated by critical point drying at 31° C. for 5 to 10 minutes, then mounted on a specimen holder for SEM and dried overnight in vacuum desiccators. In the final stage of preparation for viewing, the samples are sputter coated with gold (Edwards, model S150B Sputter Coater). Samples are viewed using SEM (Hitachi S-2500). Images are scanned directly to a computer and stored as image files for subsequent viewing. Ultramicroscopy studies help to assess the clumping effect of the drugs being tested, as well as to evaluate if there is cellular exfoliation as a result of mucomodulator use.

### Example 1

### Effect of Dextran at Various Molecular Weights on Mucus Rheology and Clearability

Low molecular weight dextran is a potential mucolytic treatment for cystic fibrosis lung disease. In pre-clinical testing, it promoted clearance by both mucociliary and airflow mechanisms, and the drug has passed a Phase 1 safety trial in normal subjects. A Phase 2 clinical trial of aerosolized low molecular weight dextran in cystic fibrosis patients has been completed, and further trials are planned. High molecular weight dextran, as evidenced from the graphs below (FIG. 2), would be less desirable as a mucolytic agent, since the primary mucolytic activity is much stronger in low molecular weight fractions; however, higher molecular weight dextrans show interesting effects in terms of predicted mucociliary and cough clearance. A reduction in log G* at 1 rad/s (upper graph) is a primary predictor of improved mucociliary clearability (6) (FIG. 2A), while a reduction in spinnability (FIG. 2B) predicts improved cough clearability. The data indicate that at some intermediate to high molecular weight level (>70,000 Daltons), there will be a dextran fraction where log G* is reduced while spinnability is increased, which will differentially affect cough and mucociliary clearability. Particularly if combined with other proposed treatments, this higher molecular weight dextran fraction should produce the desired combination of reduced cough aerosol clearance while maintaining mucociliary clearance function. Further, it will be shown in Example 5, below, that at least under some circumstances, it is possible to decrease cough aerosol formation while increasing “expectoration” (bulk mucus transport).

### Example 2

### Effect of CaCl₂ on Mucus Clumping

FIG. 3A is a scanning electron (SE) micrograph of mucus from the frog palate which had been treated with Frog Ringers (FR) solution. In mucus clearance studies on the frog palate, Frog Ringers is used as a control solution and mucus clearance or velocity is expressed relative to Frog Ringers. Mucus is mostly composed of water (95%), the remaining 5% is made up of glycoproteins linked together by various types of chemical bonds. During a cough or sneeze, air forced through the airways picks up minute mucus droplets which are expelled from the body as aerosol. The dispersion of the aerosol mist depends on the droplet size.

FIG. 3B is a SE micrograph which shows the effect of CaCl₂ (5 μL of 0.1 M solution) applied topically to the frog palate. CaCl₂ causes “clumping” of the mucus on the surface of the palate. The ciliated surface of the palate can be seen below the mucus and does not appear different from control views (FIG. 3A). This clumping action of CaCl₂ indicates that the aerosolizability of the mucus or its ability to form tiny droplets will be decreased. This action will have a positive benefit by tending to suppress the dispersion of a mucus mist during a cough or sneeze.

### Example 3

### The Effect of Five Different Mucomodulator Agents on Mucociliary Transport at Eight Different Concentrations from 10⁻⁸ to 10⁻¹ M

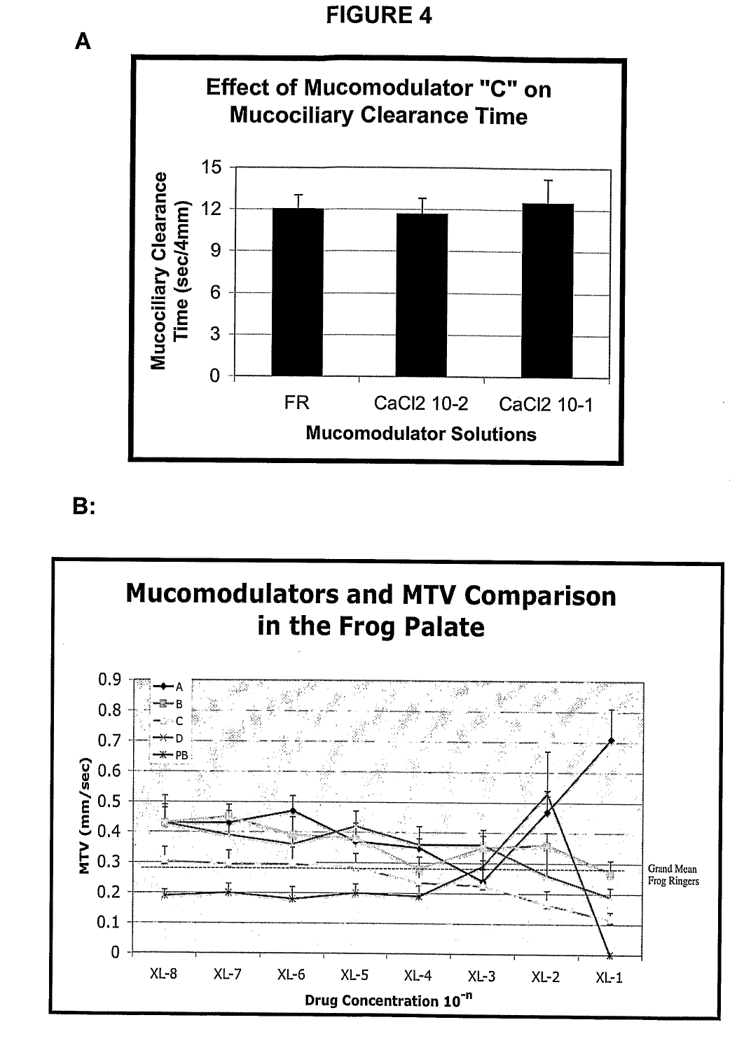

In FIG. 4A, the effect of a mucomodulating agent (CaCl₂) on mucociliary transport time is shown compared to Frog Ringers (FR) solution. Both solutions (FR and CaCl₂) were applied topically to the palate in a volume of 5 μL. Thirty seconds were allowed for dispersion of the agent on the palate, followed by the measurement of mucociliary transport time. A droplet of mucus from the palate was placed on palate toward the mouth opening. Ciliary action transported the droplet down the palate. The measurement process (timing the movement of the droplet down the palate over a set distance of 4 mm as observed through a stereomicroscope with a reticulated eyepiece) was repeated five times and averaged to give a mean transport time for each solution. It is clear from FIG. 2A, that no significant effect on mucociliary transport time was observed at either concentration of mucomodulating agent compared to Frog Ringers.

FIG. 4B shows the effects of five different mucomodulators. “A” is poly-L-arginine; “B” is sodium tetraborate (Na₂B₄O₇₀.10H₂O); “C” is calcium chloride, CaCl₂; “D” is HMW dextran (m.w. ca. 500,000); PB is polymyxin B, a cationic antimicrobial. As described above, mucomodulator solutions were applied topically to the palate in a volume of 5 μl. After 30 seconds to allow dispersion of the agent on the palate, measurements of mucociliary transport time were taken. None of the agents tested on the frog palate had any significant effect on mucociliary clearance until the highest delivered concentrations. Variations at low concentrations were due to variations in baseline frog palate mucociliary activity. Mucomodulator A (polyarginine) led to a significant increase in mucociliary clearance rate with increasing applied concentration. With polymyxin B, there was an increase in MC at an intermediate concentration, and then a sustained decrease at the highest concentration. None of the other agents tested significantly altered mucociliary clearance in this model when applied directly to the ciliated epithelium.

### Example 4

### Respiratory Tract Mucus Clearance: Model Experiments

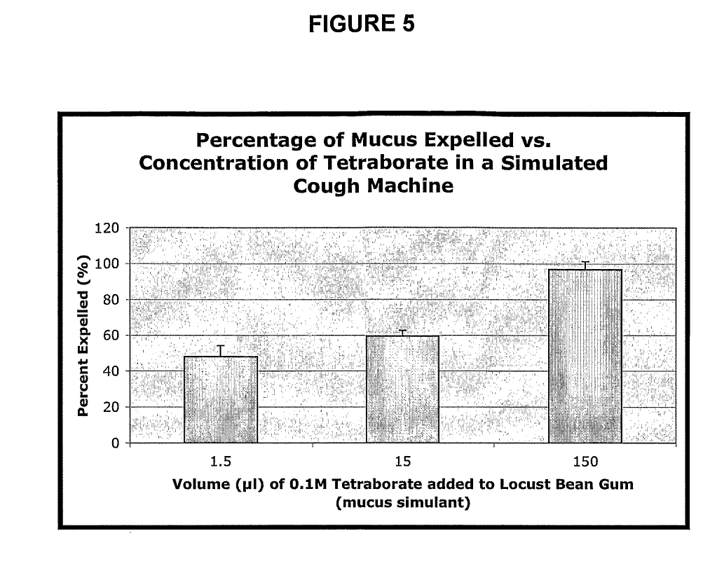

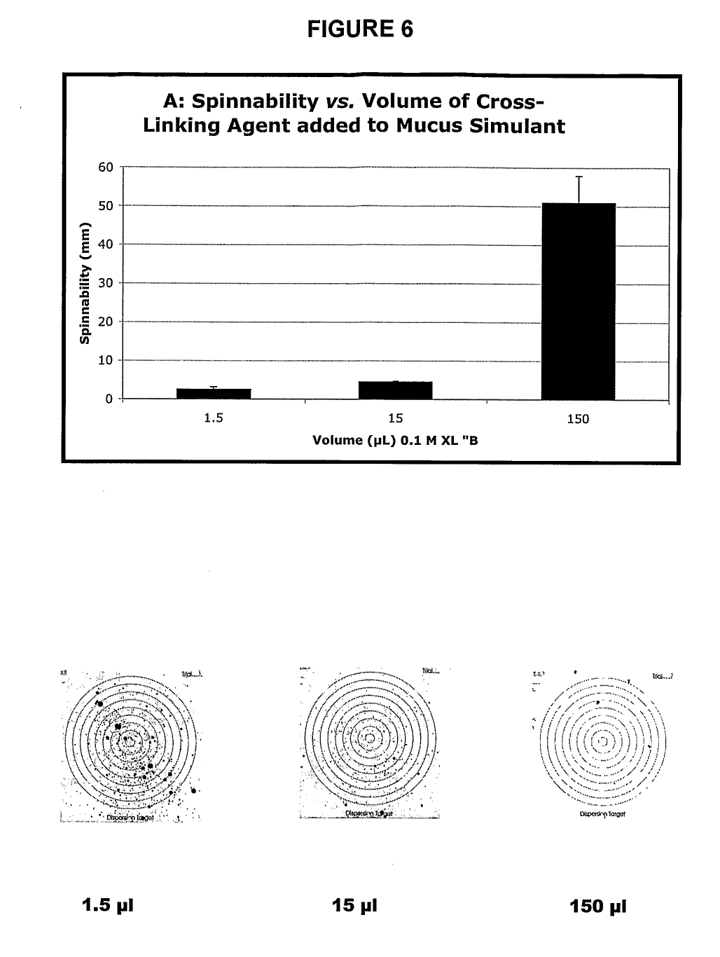

Mucus simulants (MS) were exposed to airflow in a simulated cough machine (SCM). The MS ranged from non-viscous, non-elastic substances (water—control) to MS of varying degrees of viscosity and elasticity. Mucin crosslinking was increased by adding sodium tetraborate (mucomodulator B) to viscous solutions of locust bean gum (Sigma). Mucociliary clearance of the MS was assessed on the frog palate (FIG. 5A), elasticity in the Filancemeter (FIG. 6A) and the aerosol pattern in a “bulls-eye” target (FIG. 6B). The sample loaded was weighed before and after each cough maneuver to achieve a measure of expectoration (FIG. 5B).

Mucociliary transport rate measured in the frog palate epithelial model was close to normal speed in viscoelastic samples compared to non-elastic, non-viscous or viscous-only samples of mucus simulant. Spinnability in MS ranged from 2.5±0.6 to 50.9±6.9 cm (FIG. 6A), and the amount of MS expelled from the SCM increased from 47% to 96% adding 1.5 μL to 150 μL of mucomodulator B (FIG. 5B). Concurrently, particles were inversely reduced to almost disappear from the aerosolization pattern (FIG. 6B). Mucociliary transport rates of borate-treated mucus simulants measured in the frog palate epithelial model compared to control (native frog palate mucus) showed means of 16-20 mm/min vs. 24 mm/min (FIG. 5A).

The aerosolizability of MS was modified by reducing the number of particles expelled from the SCM (FIG. 6B), while interfering minimally with its clearance on the frog palate (FIG. 5A). An unexpected and novel finding of this study is that adding crosslinks to mucus simulants led to an increase in “expectoration” or bulk clearance of the mucus simulant from the model trachea (FIG. 5B). Thus the increased crosslinking and cohesiveness in the mucus simulant indicates reduced fine aerosol formation and clearance, but enhanced bulk flow of mucus.

### Example 5

### Respiratory Airway Mucus Clearance in Anesthetized Dogs

Anesthetized, healthy, adult, mixed-breed dogs, 20-30 kg, free of respiratory tract infections or other observable respiratory tract pathology were sham exposed or randomly administered different concentrations of aerosolized HMW dextran aerosol (Mucomodulator XL D) [mol. wt. ca. 500,000] or sodium tetraborate (XL B) or normal saline (0.15M NaCl) via an endotracheal tube. The mucomodulator aerosols were delivered to the intubated, spontaneously breathing dogs by jet nebulizer aerosol (7 mL over 20 min). Three aerosol concentrations were delivered: Low=0.001M; Med 0.01M; High=0.1M.

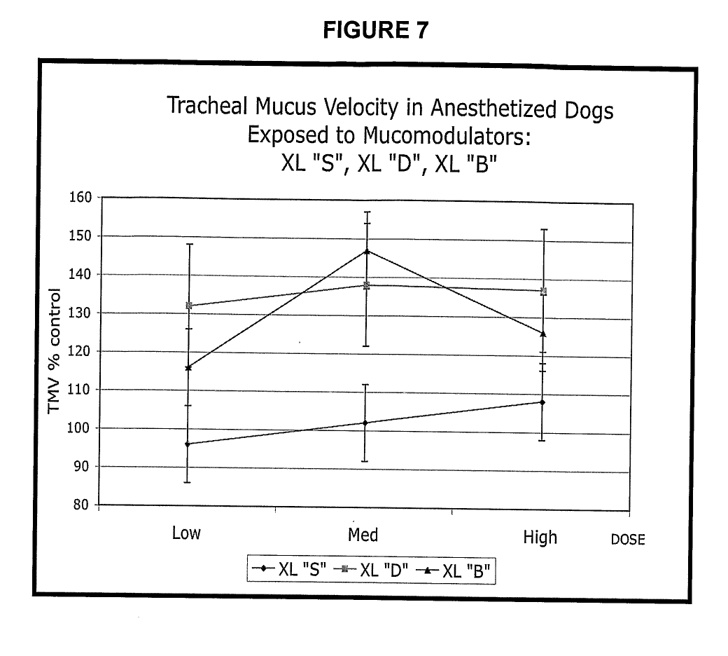

The following was assessed: cardio-respiratory pattern, tracheal mucus clearance, airway patency, and mucus viscoelasticity changes. Dogs maintained normal cardio-respiratory pattern with XL administration, and there was no evidence of mucus retention in the airways. After exposure to mucomodulators B and D, but not in sham or saline exposure, an increase in the thread formation capability (a measure of elasticity) of the tracheal mucus, and a sustained 30%-40% mean increase in tracheal mucus linear velocity compared to control was observed. The increase in mucus velocity reached an optimum for an intermediate concentration of sodium tetraborate (“Mucomodulator XL B”) (FIG. 7/Table 2). The increase over control mucociliary clearance rate was statistically significant, as was the increase over the low dose of XL B. The degree of improvement in tracheal mucociliary clearance is as good as or better than that achievable by traditional mucolytic approaches in the same animal model.

### Example 6

### Effects of Mucomodulators on Mucus Clearance and Disease Transmissibility

The effect of mucomodulator therapy in vitro, in animals and in humans can be studied. The novel mucotherapy is designed to minimize respiratory aerosol emissions while interfering only minimally with normal mucus clearance mechanisms. In the mucus samples rheological, physiological and biochemical parameters, in addition to ultrastructural parameters as visualized through scanning electron microscopy can be studied using techniques described herein in combination with those known in the art. For instance, patients having potential SARS or SARS-like symptoms can be incorporated in an open clinical trial with historical controls.

To determine acute effects, the inventors' environmental exposure ex-vivo animal model can be used, as described above. To measure mucociliary transportability, a modified frog palate technique can be used, as described above. The method of the invention can also be used to identify the effects of mucomodulators on the aerosol pattern while coughing, sneezing and speaking with or without treatment of mucomodulators as well as to estimate optimum dose and delivery system to reduce aerosol formation without compromising airway mucus clearance.

Aerosol drug delivery optimization, using bench tests and mathematical deposition models, can be used.

Mucus viscoelasticity can be determined by magnetic microrheometry, and anatomical epithelial injury by SEM. Real time ciliary beat frequency can be assessed in an optical microscope system capable of acquiring digital video sequences. Studies of aerosol pattern can be carried out in a controlled environment.

Dosing of Mucomodulators

Assessment of the use of mucomodulators under varied conditions will be studied. Therefore, studies must provide within-study flexibility in dosage. Inhaling the mucomodulator may result in a wide intersubject variation in the effective dose due to differences in inhalation techniques. Aerosol drug delivery optimization, using bench tests and mathematical deposition models, will allow determination of a recommended technique and reduce this problem. The introduction of subjects to mucomodulators can be conducted using low initial doses of the component with increases in dose being made only after adequate subject experience. The dosage information and adjustments are collected and documented for each subject.

Volunteer Exposure Phase

A group of volunteers (approximately 10) will be asked to participate. Baseline data will include: demographic data, medical history, physical exam, baseline values for critical clinical measurements indicators of response therapy, aerosol post-cough induction pattern, other relevant variables (smoking, alcohol intake, for women date of last menstrual period). The study design for this phase will be a double blind (subject and evaluator), randomized prospective controlled trial. The main variable to consider in this phase will be aerosol pattern while coughing, sneezing and speaking with or without treatment of mucomodulators, as well as to estimate optimum dose and delivery to reduce aerosol formation without compromising mucus clearance.

### CONCLUSIONS

The results indicate increased “expectoration” in the cough machine model with normal mucociliary clearance in frog palate testing as well as enhanced tracheal mucociliary clearance in anesthetized dogs, while obtaining the desired target of a significant reduction in fine aerosol formation. An increase in bulk mucus cough clearance using the mucomodulators (traditional mucothickening agents) was an unexpected and novel finding.

The present mucomodulator technology is innovative and represents a reversal in current thinking about respiratory disease management. It could potentially have an unprecedented role as a safe, efficacious therapy for airway hygiene and secretion management, as well as in prevention of droplet-spread illnesses through close person-to-person-contact and airborne transmission.

The methods and compositions of the invention can be used to improve mucus clearance in health and in disease, like, but not limited to, conditions that pose risk of mucus plugging (i.e. asthma, CF), have impaired airway secretion management (i.e. COPD, patients with spinal cord injury, neuromuscular disease, chronic sinusitis).

The methods and compositions of the invention can be used to reduce aerosol formation capability in patients; limit contagiousness of close contacts and decrease the frequency of adverse events. Although, the above-noted studies will be performed on patients with the suspected medical condition, such as SARS, the invention could be applicable to any disease transmitted by droplets or aerosol formed when coughing, sneezing and speaking, such as influenza, TB and SARS.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 1
Sputum rheological analysis - reference data
Malcolm King, PhD
Mucobiology Lab, 173 HMRC, Univ of Alberta, Edmonton, AB T6G 2S2

| | Log G*1 | logG*100 | MCI | Tan∂ 1 | Tan∂ 100 | CCI |
|---|---|---|---|---|---|---|
| NS (Mtl) | 2.19 ± .32 | 2.58 ± .33 | .91 ± .08 | .28 ± .05 | .95 ± .26 | 1.52 ± .51 |
| NS (Edm) ** | 2.12 ± .4 | 2.55 ± .33 | .94 ± .09 | .27 ± .03 | .91 ± .29 | 1.52 ± .55 |

| | | MCI | | | | | |
|---|---|---|---|---|---|---|---|
| | | (mucociliary | CCI (cough | G′, 10 rad/s | | | |
| | log G*, | clearability | clearability | (elasticity, | | σσ, 10 σσσ/σ | |
| | 1-100 rad/s | index) | index) | dyn/cm2) | | (viscosity, Poise) | |
| mean, normal | 2.39 | 0.91 | 1.52 | 107-490 | | 3.8-17.4 | |
| (±SD) | 0.33 | 0.08 | 0.51 | (1 SD range) | | (1 SD range) | |
| mean, CF | 2.80 | 0.75 | 0.91 | 195-1780 | (1 SD) | 7.4-70 | (1 SD) |
| (±SD) ‡ | 0.48 | 0.22 | 0.65 | 65-5750 | (2 SD) | 2.5-200 | (2 SD) |
| mean, CF | 3.2 | 0.7 | 0.3 | 740-2950 | (1 SD) | 28-110 | (1 SD) |
| (±SD) i | 0.3 | 0.1 | 0.4 | 370-5900 | (2 SD) | 14-225 | (2 SD) |

NS (Mtl) = Non smokers Montreal.
NS (Edm) = Non smokers Edmonton
tan∂ = mechanical loss
tangent = “viscosity/elasticity”
log G*, 1-100 rad/s = mucus rigidity index = vector sum of “viscosity + elasticity” = mechanical impedance (log scale) averaged over measurement frequency range (this is the best overall representation of mucus viscoelasticity)
MCI = mucociliary clearability index = 1.62 − 0.22*logG*1 − 0.77*tan∂1 (predicts the normalized clearance rate on frog palate ciliated epithelium)
CCI = cough clearability index = 3.44 − 1.07*logG*100 + 0.89*tan∂100 (predicts the normalized clearance by airflow in simulated cough machine) (minimum value normally constrained to zero)
G′, 10 rad/s = elasticity = shear storage modulus, measured at 10 rad/s (1.7 Hz)
σ′, 10 rad/s = viscosity = loss modulus/frequency, at 10 rad/s normal data adapted from Jeanneret-Grosjean et al., Am Rev Respir Dis 1988; 137: 707-710
** Zayas et al. Am Rev Respir Dis 1990; 141: 1107-1113
CF data from meta-analysis of control/baseline sputum from adult patients
‡ (Tomkiewicz et al. Am Rev Respir Dis 1993; 148: 1002);
i (App et al. Chest 1998; 114: 171)

TABLE 2

| | TMV, mm/min | | | | |
|---|---|---|---|---|---|
| Mucomodulator | Control | Low | Med | High | n |
| XL “S” | 2.95 ± 1.4 | 3.1 ± 2.2 | 3.3 ± 2.46 | 3.15 ± 1.45 | 38 |
| XL “D” | 3.6 ± 2.24 | 4.43 ± 3.83 | 3.77 ± 1.65 | 3.85 ± 2.2 | 36 |
| XL “B” | 3.42 ± 0.94 | 3.88 ± 1.66 | 4.96 ± 1.99*† | 4.3 ± 1.53 | 30 |

S = saline/sham;
D = HMW dextran;
B = sodium tetraborate
Aerosol concentrations: Low = 0.001M; Med = 0.01M; High = 0.1M
Mucomodulators delivered to spontaneously breathing anesthetized dogs by jet nebulizer aerosol (7 mL over 20 min).
*= sig. diff. from saline control (p < .05);
† = sig. diff. from low dose.

### FULL CITATIONS FOR REFERENCES REFERRED TO IN THE SPECIFICATION

1. King M, Brock G, Lundell C. Clearance of mucus by simulated cough. J Appl Physiol 1985; 58: 1776-1782.
2. Jeanneret-Grosjean A, King M, Michoud M C, Liote H, Amyot R. Sampling technique and rheology of human bronchial mucus. Am Rev Respir Dis 1988; 137: 707-710.
3. Rubin B K, Ramirez 0, King M. Mucus-depleted frog palate as a model for the study of mucociliary clearance. J Appl Physiol 1990; 69: 424-429.
4. Tomkiewicz R P, App E M, Zayas J G, Ramirez O, Church N, Boucher R C, Knowles M R, King M. Amiloride inhalation therapy in cystic fibrosis: Influence on ion content, hydration and rheology of sputum. Am Rev Respir Dis 1993; 148: 1002-1007.
5. Hardy J G, Everard M, Coffiner M, Fossion J. Lung deposition of a Nacystelyn metered dose inhaler formulation. J Aerosol Med 1993; 6:37-44.
6. King M, Rubin B K. Rheology of airway mucus: Relationship with clearance function. Chapter 7 of: Takishima T, Shimura S, eds. Airway Secretion: Physiological Bases for the Control of Mucus Hypersecretion (Lung Biology in Health and Disease Series) New York: Marcel Dekker, 1994, 283-314.
7. Feng W, Garrett H, Speert D P, King M. Improved clearability of cystic fibrosis sputum with dextran treatment in vitro. Am J Respir Crit. Care Med 1998; 157: 710-714.
8. King M, Festa E. The evolution of the frog palate model for mucociliary clearance. In: Baum G, ed. Cilia, Mucus and Mucociliary Interactions. New York: Marcel Dekker, 1998, 191-201.
9. Finlay W H, Lange C F, King M, Speert D P. Lung delivery of aerosolized dextran. Am J Respir Crit. Care Med 2000; 161: 91-97.
10. Vanderbist F, Wery B, Baran D, Van Gansbeke B, Schoutens A, Moes A J. Deposition of nacystelyn from a dry powder inhaler in healthy volunteers and cystic fibrosis patients. Drug Dev Ind Pharm 2001; 27:205-12.
11. King M, Rubin B K. Pharmacological approaches to discovery and development of new mucolytic agents. Advanced Drug Delivery Reviews 2002; 54: 1475-1490.
12. King M. Mucus, mucociliary clearance and coughing. In: Bates D V. Respiratory Function in Disease, 3rd ed. Philadelphia: Saunders, 1989: 69-78.
13. King M. Role of mucus viscoelasticity in cough clearance. Biorheology 1987; 24: 589-597.
14. Zayas J G, Man G C W, King M. Tracheal mucus rheology in patients undergoing diagnostic bronchoscopy: Interrelations with smoking and cancer. Am Rev Respir Dis 1990; 141: 1107-1113.
15. Wanner A, Salathé M, O'Riordan T G. Mucociliary clearance in the airways. Am J Respir Crit. Care Med 1996; 154: 1868-1902.
16. King M, Rubin B K. Mucus physiology and pathophysiology: Therapeutic aspects. Chapter 13 of: Derenne J P, Whitelaw W A, Similowski T, eds. Acute Respiratory Failure in COPD (Lung Biology in Health and Disease Series). New York: Marcel Dekker, 1996: 391-411.
17. King M, Rubin B K. Mucus controlling agents: Past and present. In: Rau J L, ed. Aerosolized Drugs for the Respiratory Tract. Respir Care Clinics N Amer 1999: 575-594.
18. King M, Rubin B K. Pharmacological approaches to discovery and development of new mucolytic agents. Advanced Drug Delivery Reviews 2002; 54: 1475-1490.
19. King M. Magnetic microrheometer. In: Braga P C, Allegra L, eds. Methods in Bronchial Mucology. New York: Raven Press, 1988, 73-83.

1. A method of enhancing mucus function comprising administering an effective amount of a mucothickening agent to a subject in need thereof.

2. The method according to claim 1, wherein the mucus function is selected from respiratory tract mucus clearance, epithelial protection, ion exchange and nutrient intake.

3. The method according to claim 2, wherein the respiratory tract mucus clearance is mucociliary clearance and/or cough clearance.

4. The method according to claim 1, wherein the enhancing of mucus function is improving physical and/or biochemical properties of the layer of mucus lining the respiratory system, digestive system, urinary tract and/or the reproductive system.

5. The method according to claim 4, wherein the enhancing of mucus function is improving physical and/or biochemical properties of the layer of mucus lining the digestive system to enhance nutrient intake processes, epithelium protection and drug absorption.

6. The method according to claim 4, wherein the enhancing of mucus function is improving physical and/or biochemical properties of the layer of mucus lining the reproductive system to enhance conception and/or inhibit, transmission of sexually transmitted diseases (STD).

7. The method according to claim 1, wherein the enhancing of mucus function is for inhibiting aerosolization and/or transmission of an airborne disease.

8. The method according to claim 7, wherein the airborne disease is selected from, Severe Acute Respiratory Syndrome (SARS), tuberculosis and influenza.

9. The method according to claim 7, wherein the inhibition of aerosolization and/or transmission of an airborne disease occurs by decreasing aerosolizable respiratory secretions in a way to minimize any adverse effect on mucus clearance, optionally in combination with enhancement of mucus clearance.

10. The method according to claim 1, wherein the enhancing of mucus function is for treating a condition related to thin mucus or cilia malfunction or non-function.

11. The method according to claim 10, wherein the conditions related to thin mucus or cilia malfunction or non-function include any condition of impaired airway secretion management.

12. The method according to claim 11, wherein the conditions are selected from cystic fibrosis, asthma, Chronic Obstructive Pulmonary Disease (COPD), spinal chord injury, neuromuscular disease and those conditions or diseases requiring assisted mechanical ventilation.

13. (canceled)

14. The method according to claim 1, wherein the mucothickening agent is any agent that promotes the formation of one or more of the following in mucus: covalent bonds, ionic bonds, hydrogen bonds, van der Waals' forces, intermingling or extracellular DNA & F-actin network.

15. The method according to claim 14, wherein the mucothickening agent is selected from one or more of high molecular weight polysaccharides, salts of tetrafunctional anions, salts of divalent cations and polycationic agents, and other pharmaceutically acceptable salts thereof.

16. The method according to claim 15, wherein the mucothickening agent is selected from one or more of dextran, sodium tetraborate, calcium chloride, magnesium chloride, polylysine, polyarginine and polymyxin B.

17-25. (canceled)
